(19) United States
(12) Patent Application Publication
(10) Pub. No.: US 2017/0362157 A1
(43) Pub. Date: Dec. 21, 2017

(54) METHOD FOR RECOVERING SUCCINIC ACID CRYSTALS USING SURFACTANTS DURING CRYSTALLISATION, AND RESULTING CRYSTALS

(71) Applicant: Roquette Freres, Lestrem (FR)

(72) Inventors: Pierrick Duflot, La Couture (FR); Pierre Lanos, La Bassee (FR); Baptiste Boit, La Gorgue (FR); Frédérick Dehay, Laventie (FR); Laurent Rossi, Arras (FR)

(73) Assignee: Roquette Freres, Lestrem (FR)

(21) Appl. No.: 15/529,648

(22) PCT Filed: Nov. 26, 2015
(86) PCT No.: PCT/FR2015/053226

§ 371 Date: May 25, 2017

(30) Foreign Application Priority Data
Nov. 26, 2014 (FR) 14 61470

(51) Int. Cl. C07C 51/43; C07C 55/10

(52) U.S. Cl. CPC C07C 51/43; C07C 55/10

(57) ABSTRACT

The invention relates to a method for producing succinic acid crystals, comprising double crystallisation and the use of at least one surfactant during the first crystallisation step. The invention also relates to the succinic acid crystals obtained from a fermentation medium, characterised in that they have a colour index b, measured in the reference system L, a, b, that is less than or equal to 1.00, preferably less than or equal to 0.90, and, better still, less than or equal to 0.80.

### FIELD OF THE INVENTION

A subject of the present invention is an improved process for recovering succinic acid crystals, one of the originalities of which lies in double crystallization and the use of surfactants during the first crystallization step. Very advantageously, at the end, crystals are obtained with a color index b in the reference system (L, a, b) of less than or equal to 1.00, which is a guarantee of products having excellent stability, that can be used to produce polymer materials without impairing the coloring thereof.

### PRIOR ART

Succinic acid (or butanedioic acid) is an organic acid with two carboxyl groups, of semi-expanded formula COOH—CH2—CH2—COOH, which today has many applications in the cosmetics, food-processing, pharmaceutical and textile fields and in the plastics industry. By way of example for the latter application, it can be used as a synthesis intermediate in the production of 1,4-butanediol, tetrahydrofuran and gamma-butyrolactone.

Initially, succinic acid was a product to synthesized by virtue of processes centered around raw materials of fossil origin. Alternatives to these methods were then developed, involving biobased products. In this respect, succinic acid can today be produced from renewable raw materials: in the case in point, by means of fermentation processes.

Various microorganisms are known for their capacity to produce succinic acid according to this pathway, such as Actinobacillus succinogenes, Mannheimia succiniciproducens, Escherichia coli, or Aspergillus niger and Saccharomyces cerevisiae. This being the case, the fermentation products contain substantial amounts of impurities (biomass debris, sugars, amino acids, trace elements, salts, etc.) which are coloration precursors capable, by their presence, even in trace amounts in the final product, of influencing the quality of the purified succinic acid, and consequently, the quality of the polymer synthesized from the purified succinic acid.

Those skilled in the art are today aware of many processes for producing succinic acid which uses various purification and/or decoloring steps.

Mention may be made of processes using solvents. This is, for example, the case with the method described in U.S. Pat. No. 6,265,190 which teaches the recovery of succinic acid by addition of ammonium sulfate in a fermentation medium with a high concentration of succinic ions. Methanol is then used to purify the succinic acid obtained.

Nanofiltration is also a recognized technique for purifying succinic acid crystals: document CN 101475464 is an example thereof.

Processes using ion exchange resins or activated carbon are also known. Thus, U.S. Pat. No. 5,168,055 teaches the reaction between sulfuric acid and a fermentation medium rich in calcium succinate, so as to jointly produce calcium sulfate and succinic acid. The latter is purified by virtue of a strong cationic resin and a weak anionic resin. Document WO 2013/169447 describes the use of non-functionalized resins. Document WO 2009/082050 proposes treating the fermentation medium before crystallization, by means of activated carbon.

Two patent applications filed by the applicant company are also an illustration of the methods mentioned in the previous paragraph: patent applications WO 2011/064151 and WO 2013/144471. It should be noted that the processes described here combine the use of activated carbon with the use of resins, while at the same time relying on double crystallization.

There is also an area of the prior art which combines nanofiltration and techniques based on ion exchange resins and/or activated carbon. In this collection, mention may be made of documents US 2012/0289742, CN 101215583, US 2010/0317891 and WO 2014/106532.

This being the case, to the best of the applicant company's knowledge, none of the existing methods for the production of succinic acid is capable of resulting in a product which has a level of coloring impurities that is sufficiently low so as to in the end obtain polymers of which the color is not modified.

More specifically, the applicant company has already shown, in patent application WO 2013/144471, that an appropriate colorimetric measurement reflects perfectly well the level of impurities responsible for discoloration of the succinic acid, this discoloration being entirely linked to a discoloration of the final polymer produced with this succinic acid. It is recalled that any colorimetric measurement is based on the opposite colors theory which specifies that the responses of the cones (the cells of the retina of the human eye that are responsible for color vision) to the colors red, green and blue are recombined into opposite “black-white”, “red-green” and “yellow-blue” signals when transmitted to the brain by the optic nerve. This measurement is based in particular on the color scales widely used in the food industries and the polymer industries, called the Hunter “L”, “a”, “b” scales. The term “reference system (L, a, b)” is also used.

In the abovementioned patent application, it was in particular demonstrated that an index “b” of 1.1 (measured in the reference system L, a, b) was at best achieved for succinic acid crystals, which themselves made it possible to produce a polymer of PBS (polybutylene succinate) type having an “acceptable colorimetric quality”, the latter being measured through the yellow index “YI” (according to ASTM standard D1925).

However, following numerous studies, the applicant company has successfully developed a particularly simple process which results in succinic acid crystals that have a colorimetric index “b”, as measured in the reference system (L, a, b), which has never yet been achieved, namely less than or equal to 1.00 and even in certain cases less than or equal to 0.90 and very preferentially less than or equal to 0.80.

This process is based in particular on the introduction of surfactants during a first crystallization step. Said step is followed by subsequent steps of dissolving the crystals formed, of purifying the solution obtained, in particular by treatment with activated carbon and/or ion exchange resins, by a second crystallization step, and then drying and cooling the crystals obtained.

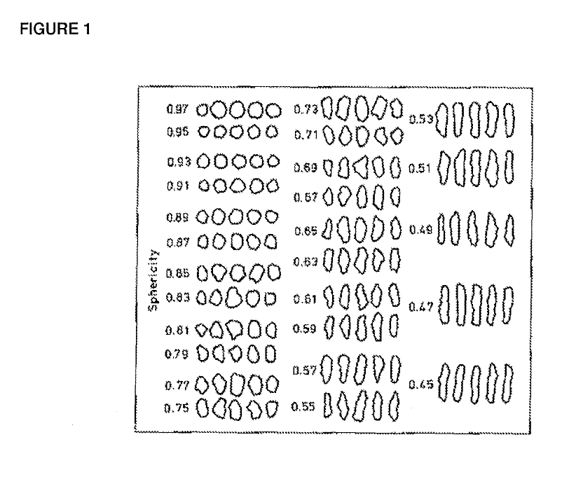

Moreover, the process according to the present invention implements 2 crystallization steps. Advantageously, the first crystallization step results in crystals in the shape of “balls”, that it will be seen improve certain aspects of the process in question. The term “balls” is in particular used as opposed to “needles” which are the other most widespread shape in which succinic acid crystals can be obtained. Nevertheless, said balls can be defined positively through a “sphericity” index. This index is, throughout the present application, determined visually on the basis of the standard definition of shape by Rittenhouse (“Agglomeration in industry”, W. Pietsch, p.600 vol. 2, 2005 Wiley-VCH), which consists in associating a sphericity index with a visual assessment of the shape of the particles observed. FIG. 1 reveals in particular the grid for evaluation and assignment of the grades according to this test.

In the case of the present invention, the use of surfactant results in the formation of ball-shaped crystals at the level of the first crystallization step, this shape being subsequently preserved in the process. The term “ball” is intended to mean here crystals having a sphericity index, as measured according to the Rittenhouse test, at least equal to 0.70, preferentially at least equal to 0.75, very preferentially at least equal to 0.85.

In point of fact, it is particularly advantageous to have ball-shaped crystals and not needle-shaped crystals, insofar as it is easier to separate the former from the crystallization mother liquors during a centrifugation or filtration step. This is in particular reported in the document “Chirality in Industry II: Developments in the Commercial Manufacture and Applications of Optically Active Compounds” (A. N. Collins, G. N. Sheldrake, J. Crosby, John Wiley & Sons, 1997-p 125).

It is also known that needle-shaped crystals are more difficult to rinse after crystallization and the separation between said crystals and the stream of mother liquors: this rinsing step consists in removing the residual mother liquors at the surface of the crystals. In this regard, reference may be made to the documents “Handbook of Industrial Drying” (Arun S. Mujumdar, 4th Edition, CRC Press, p 1273, 64.1.5 Crystal purity) and Crystal Shape Enhancement: a Processing Solution to a Product Problem (Snyder, R. C., Studenar, S., Doherty, M. F., AlChE 2006 Annual Meeting).

Furthermore, needle-shaped crystals have a tendency to exhibit more impurities in the form of embedded mother liquors, that is to say mother liquors which are physically trapped in the crystal. This thus results in a poorer purification capacity that the surfactant makes it possible to improve (Handbook of Industrial Crystallization, 2^{nd} Edition, Allan S. Myerson, p 259).

From the viewpoint of the prior art, such a result is particularly surprising. Indeed, document WO 01/07389 is known, which teaches the use of surfactants at the time of succinic acid crystal formation. It is clearly explained in said document that the use of surfactants then results in the formation of needle-shaped crystals and not ball-shaped crystals.

The applicant company has also shown that the addition of surfactant enables recycling of part of the crystallization and washing mother liquors at the top of the first crystallization, thereby improving the succinic acid recovery yield and making it possible to obtain a “b” in accordance with the invention. The recovery yield is defined as the ratio of the weight of succinic acid crystals obtained after drying to the weight of succinic acid contained in the acidified fermentation liquor (before step b)). This yield may also be expressed as a percentage.

Thus, not only did the applicant company have to select the starting process ad hoc from all the methods thus far available: that described in documents WO 2011/064151 and WO 2013/144471, based on double crystallization, it then went against what the prior art was teaching: using surfactants in a succinic acid crystallization step in order to improve the quality thereof by very considerably decreasing the number of coloring impurities, but also in order to favor the formation of ball-shaped crystals and not needle-shaped crystals.

It has also shown that the surfactants have to be used during the first crystallization step and not during the second. The use of surfactants during the second crystallization step in fact again generates needles and unsatisfactory “b” values. Finally, it has demonstrated that the products obtained have a “b” index (in the reference system L, a, b) lower than those obtained through the prior art processes involving only a single crystallization and the combination of various steps based on ion exchange resins and activated carbon. It has shown that this index is less than or equal to 1.00, advantageously less than or equal to 0.90 and very preferentially less than or equal to 0.80.

### SUMMARY OF THE INVENTION

Thus, a first subject of the present invention consists of a process for producing succinic acid crystals from a fermentation medium containing succinic acid, comprising the steps of:
a) bringing the fermentation medium to a pH of between 1.0 and 4.0,
b) crystallizing the succinic acid from the fermentation medium resulting from step a) so as to form succinic acid crystals, separating the succinic acid crystals from the crystallization mother liquors and then washing the obtained crystals with water,
c) dissolving the succinic acid crystals obtained following step b) in water at a temperature between 30° C. and 70° C. so as to obtain a solution containing dissolved succinic acid,
d) purifying the succinic acid solution obtained in step c) using a treatment on activated carbon and on ion exchange resin,
e) crystallizing the succinic acid contained in the solution obtained in step d) so as to form succinic acid crystals, subsequently separating the succinic acid crystals from the crystallization mother liquors and then washing the obtained crystals with water,
f) drying the succinic acid crystals to a moisture content of less than 0.5% and cooling them to a temperature below 30° C.,
   characterized in that at least one surfactant is introduced before and/or during step b). Preferably, said surfactant is introduced during step b).

Throughout the present application, the term “surfactant” is intended to mean a compound which modifies the surface tension between two surfaces.

Said surfactant is preferentially chosen from non-ionic surfactants, and preferentially from polysorbates having an HLB greater than 15, for example of Tween 20 type, from surfactants based on an alkylene oxide block copolymer, for example of Erol 18 (OUVRIE PMC) or Supra NS 1342 (HYPRO-Food) type, preferentially block copolymers of propylene oxide and ethylene oxide, and from those having in particular anti-foaming properties without however this list being exhaustive.

The first step a) of the process according to the invention thus consists in bringing the fermentation medium to a pH of between 1.0 and 4.0. The pH can in particular be brought to a value of between 1.5 and 3.5 and preferentially between 1.5 and 3.0.

The fermentation medium typically contains bacteria chosen from bacterial strains of the Mannheimia, Anaerobiospirillum, Bacillus or Escherichia genus, or from fungal cells. The fungal strains can be chosen from Saccharomyces cervisiae, Saccharomyces uvarum, Saccharomyces bayanus, Schizosaccharomyces pombe, Aspergillus niger, Penicillium chrysogenum, P. symplissicum, Pichia stipidis, Kluyveromyces marxianus, K. lactis, K. thermotolerans, Yarrowia lipolytica, Candida sonorensis, C. glabrata, Hansenula polymorpha, Torulaspora delbrueckii, Brettanomyces bruxellensis, Rhizopus orizae, lssatchenkia orientalis or Zygosaccharomyces bailii. The bacterial strains can be chosen from Mannheimia succiniciproducens, Anaerobiospirfflum succiniciproducens, Bacillus amylophylus, B. ruminucola or col.

The fermentation medium consists of any fermentation medium capable of generating succinic acid. It can in particular contain a carbon source, such as glucose, fructose, galactose, xylose, arabinose, sucrose, lactose, raffinose or glycerol.

The fermentation may be of aerobic or anaerobic nature, or under particular oxygen deficiency conditions, or result from a combination of these conditions, as described in document WO 2009/083756.

Usually, a neutralizing agent is then introduced into the fermentation medium, for instance potassium hydroxide or sodium hydroxide.

The regulation of the pH in the desired zone, that is to say between 1.0 and 4.0, preferentially between 1.5 and 3.5, very preferentially between 2.0 and 3.0, can be carried out by any of the means available to those skilled in the art and capable of bringing about a change in pH in this field. Mention may be made of bipolar electrodialysis in combination with strong or weak cationic resins (respectively, for example, resins of the divinylbenzene polystyrene (DVB) type with sulfonic groups or resins based on maleic and fumaric acids), weak or strong cationic resins used alone, or acidification by direct addition of hydrochloric acid or of sulfuric acid.

At the end of step a), the acidified fermentation liquor typically has a dry matter content of between 5% and 10% by weight. This liquor is then concentrated, by evaporation, to a dry matter content of between 15% and 50%, preferentially between 20% and 40% by weight, very preferentially between 30% and 35% by weight.

The second step, b), of the process according to the invention consists in crystallizing the succinic acid from the fermentation medium resulting from step a) so as to form succinic acid crystals, then in separating the succinic acid crystals from the crystallization mother liquors and finally in washing the acid crystals with water. The crystallization takes place according to any of the methods well known to those skilled in the art, either batchwise or continuously and by cooling, in particular by direct contact with the crystallization medium or by flash cooling.

The separation between the crystals and the mother liquors can be carried out by any of the techniques well known to those skilled in the art, and in particular by filtration or centrifugation.

The washing, for its part, is carried out with water, preferentially with demineralized water, at a temperature between 15° C. and 25° C., preferentially at approximately 20° C.

In addition, it is advantageous to recycle part of the crystallization and washing mother liquors to the top of step b). This recycling relates to at most 70% by weight of the total mother and rinsing liquors, more preferentially to 20% to 60%, more preferentially to 30% to 50% by weight thereof.

This step is in particular characterized in that at least one surfactant is introduced during this step and/or before this step, that is to say directly into the fermentation medium before crystallization. The surfactant(s) is (are) introduced batchwise or continuously by means of a metering pump. The amount of surfactant is preferentially between 100 ppm and 5000 ppm, preferably between 500 ppm and 3000 ppm, more preferentially between 1000 ppm and 2000 ppm, relative to the weight of succinic acid solution originating from step a) and reaching the top of step b). As early as this step b), it should be noted that the succinic acid crystals obtained generally have a sphericity index, as measured according to the Rittenhouse test, at least equal to 0.70, preferentially at least equal to 0.75, and very preferentially at least equal to 0.85.

The third step, c), of the process according to the invention consists in dissolving the succinic acid crystals obtained following step b) in water, preferably demineralized water, at a temperature between 30° C. and 70° C. so as to obtain a solution containing dissolved succinic acid. The succinic acid is dissolved so as to obtain a dry matter content of between 5% and 50% by weight of the total weight of said solution, preferentially between 10% and 20%.

The fourth step, d), of the process according to the invention consists in purifying the succinic acid solution obtained in step c) using a treatment on activated carbon and on ion exchange resin. The carbon in question may be in powder form, or granular form, more preferentially in granular form on a column operating as a fixed bed or a moving bed. The ion exchange (IEX) resins may be strong cationic resins, such as divinylbenzene polystyrene (DVB) resins of strong cationic type with sulfonic groups and of weak anionic type with quaternary or tertiary amine groups.

The fifth step, e), consists in crystallizing the succinic acid contained in the solution obtained in step d) in order to recover the succinic acid in the form of crystals and in separating succinic acid crystals and crystallization mother liquors and washing the obtained succinic acid crystals with water.

The crystallization, separation and washing steps can be carried out according to the same recommendations as for the first crystallization step. In addition, it is advantageous to recycle all of the crystallization and washing mother liquors of step e) to the top of step b).

The sixth step, f), consists in drying the succinic acid crystals to a moisture content of less than 0.5%, preferentially less than 0.4%, very preferentially less than 0.3% by weight of water relative to the dry weight of succinic acid, and in cooling them to a temperature below 30° C., preferentially below 25° C.

The process according to the invention makes it possible to obtain the succinic acid crystals described hereinafter.

Thus, another subject of the present invention consists of succinic acid crystals obtained from a fermentation medium containing succinic acid, characterized in that they have a color index b, measured in the reference system L, a, b, of less than or equal to 1.00, preferentially less than or equal to 0.90, very preferentially less than or equal to 0.80.

This composition may in particular be a powder consisting of succinic acid crystals having the abovementioned index “b” value.

Throughout the present application, the parameter “b” is measured as follows:
1) a crystalline powder of succinic acid having at least 1% of residual water content is prepared, in particular by drying the powder in such a way as to obtain this content,
2) a sample of said crystalline powder is placed in an oven at 220° C. for 2 h,
3) the crystalline powder thus treated is milled and sieved, in such a way that its particle size distribution is the following, as determined on a Retsch vibrating sieve:
   from 0% to 10%, preferably from 4% to 6%, by weight of the particles having a size greater than 500 μm,
   from 20% to 40%, preferably from 25% to 35%, by weight of the particles having a size of between 200 pm and 500 μm,
   from 50% to 75%, preferably from 55% to 70%, by weight of the particles having a size of less than 200 μm,
4) the color of the milled and sieved powder is measured in a spectrocolorimeter and the mean value of the index “b” is determined.

The measurement is carried out 10 times on the same sample, thereby giving an uncertainty of +/−0.05 on the result. Said measurement is carried out on a spectrocolorimeter which enables the reflection of the wavelength between 400 nm and 700 nm to be measured, for instance the Dataflash 100 sold by the company Datacolor. (Measurement aperture: 9 mm in diameter; reading illuminant: C2 Deg).

The crystals can in particular have a sphericity index, as measured according to the Rittenhouse test, at least equal to 0.70, preferentially at least equal to 0.75, and very preferentially at least equal to 0.85.

The composition according to the invention can comprise at least 50% by number of said succinic acid crystals, advantageously at least 70%, preferentially at least 90%. Most preferentially, the composition consists essentially of the crystals according to the invention.

Advantageously, the crystals also have a reducing sugar content of less than 20 ppm, preferentially less than 10 ppm, relative to the total weight of anhydrous crystals. This content is generally greater than 0.1 ppm. The sugars that are typically found are glucose, mannose, trehalose, isomaltose, maltose, maltulose, gentobiose and panose. The measurement is carried out according to the 2004 technical brochure of the company Dionex “Analysis of Carbohydrates by High-Performance Anion-Exchange Chromatography with Pulsed Amperometric Detection (H PAE-PAD)”.

The examples which follow make it possible to better illustrate the application, without however limiting the scope thereof.

### EXAMPLES

### Example 1

### Fermentation

A fermentation medium is prepared under the strict conditions of example 1 of document WO 2011/064151. The only things that differ are the devices used: the pre-culture step is carried out on a Puntbus 6 I reactor, the growth phase on a 7 m³ fermenter and the production phase on two 70 m³ fermenters. The fermentation liquor flow rate is then approximately 1.5 m³/h.

The separation of the biomass and the fermentation liquor is carried out by microfiltration. The latter is carried out batchwise at a temperature of 80° C., followed by a diafiltration step. The module is equipped with 2 “Kerasep” ceramic casings of 25 m², having a porosity equal to 0.1 pm. The mean permeate flow rate is approximately 2 m³/h with a transmembrane pressure of approximately 1 bar.

### Acidification Step a)

The fermentation liquor is treated on weak cationic resin of Amberlite IRC 747 type at a flow rate of 2 BV/h at 60° C. in order to achieve a divalent ion concentration of less than 5 ppm. The resins are regenerated after having passed over a volume of fermentation liquor of between 30 and 40 times the resin volume.

The solution obtained is then acidified on an Aqualyzer® EDBM EUR40 BPED (bipolar electrodialysis) module sold by the company Eurodia, at a pH of approximately 3.5.

The solution is then treated on a strong cationic resin of Purolite C150 type at a flow rate of 2 BV/h and at a temperature of 40° C. in order to achieve a pH of 2.0. The resins are regenerated after having passed over a volume of fermentation liquor of between 15 and 20 times the resin volume.

### First Crystallization: Step b)

The acidified solution is concentrated on a forced-circulation vacuum plate evaporator sold by the company Alfa Laval, to a concentration of 35% by dry matter and at a temperature of 80° C. It is then continuously crystallized by flash cooling on 2 stages each composed of an external-loop vacuum crystallizing dish sold by the company Gea Kestner. The vacuum in the crystallizing dishes is fixed so as to obtain a temperature on the first stage of 40° C. and on the second stage of 20° C. The residence time is approximately 5 h.

In the case of the use of the surfactant, the latter is continuously introduced by means of a metering pump at a flow rate equal to 1200 ppm relative to the flow rate of solution which feeds the crystallizing dish. The surfactant chosen is Erol 18 sold by the company PMC Ouvrie.

The crystalline mass is then separated on a batch centrifuge of SC 1200 type sold by the company Robatel in order to recover the succinic acid crystals. During this step, the crystals are washed with demineralized water at 20° C. with an amount equal to 1 kg/kg of crystals.

Between 0% and 70% by weight of the crystallization and washing mother liquors recovered on the centrifuge are recycled to the top of the evaporator.

### Crystal Dissolving Step c)

The succinic acid crystals are dissolved at a temperature of 45° C. with demineralized water so as to obtain a solution at 10% dry matter content.

### Purification Step d)

The step of treatment on activated carbon is carried out with a granular carbon of Chemviron CPG LF 12×40 type on a column operating as a fixed bed. The flow rate of solution in the column is fixed at 0.5 BV/h and the volume of solution treated before renewal of the bed is variable depending on the operating conditions and the quality desired. It is between 40 and 500 times the volume of carbon of the column.

The step of treatment on ion exchange resins is carried out at a flow rate of 2 BV/h at 60° C. on a strong cationic resin of Dowex 88 type, then on a weak anionic resin of Lanxess Lewatit S4528 type. The resins are regenerated after having passed over a volume of solution equal to 40 times the resin volume.

### Second Crystallization: Step e)

The purified succinic acid solution is concentrated on a falling-film vacuum evaporator sold by the company Wiegand, to a concentration of 30% by dry matter and at a temperature of 80° C. It is then continuously crystallized by flash cooling on 2 stages each composed of an external-loop vacuum crystallizing dish sold by the company Gea Kestner. The vacuum in the crystallizing dishes is fixed so as to obtain a temperature on the first stage of 40° C. and on the second stage of 20° C. The residence time is approximately 7 h.

The crystalline mass is then separated on a batch centrifuge of SC 1200 type sold by the company

Robatel in order to recover the succinic acid crystals. During this step, the crystals are washed with demineralized water at 20° C. with an amount of crystals equal to 1 kg/kg.

All of the crystallization and washing mother liquors are recovered on the centrifuge and recycled to the top of the evaporator at the level of step b).

### Drying Step f)

The product is dried on a rotary dryer so as to obtain a residual moisture content equal to 0.3% by weight of water relative to the total weight of product, then cooled on a fluidized bed at a temperature of 25° C.

### Example 2

### Fermentation

A fermentation medium is prepared under the strict conditions of example 5 of document WO 2011/064151. The only things that differ are the devices used: the pre-culture step is carried out on a Puntbus 6 I reactor, the growth phase on a 7 m³ fermenter and the production phase on two 70 m³ fermenters.

The separation of the biomass and the fermentation liquor is carried out by microfiltration. The latter is carried out batchwise at a temperature of 80° C., followed by a diafiltration step. The module is equipped with 2 “Kerasep” ceramic casings of 25 m², having a porosity equal to 0.1 pm. The mean permeate flow rate is approximately 2 m³/h with a transmembrane pressure of approximately 1 bar.

### Acidification Step a)

The fermentation liquor is then treated on a strong cationic resin of Purolite C150 type at a flow rate of 2 BV/h and at a temperature of 40° C. in order to achieve a pH of 2.0. The resins are regenerated after having passed over a volume of fermentation liquor of between 15 and 20 times the resin volume.

All of the other steps b) to f) are carried out as described in example 1.

### Example 3

This example corresponds to the performing of tests according to or outside the invention (with or without surfactant), in a double crystallization process.

Six tests were carried out, so as to evaluate the influence of 2 parameters: the value of the ratio of the volume of solution treated to the volume of carbon in step c) (“Bed Volume” or BV=Volume of solution/volume of activated carbon) and the % of crystallization mother liquors recycled at the level of step b).

Tests Nos. 1 to 3 are carried out according to the protocol given in example 1 and without surfactant.

Tests Nos. 4 to 6 are carried out according to the protocol given in example 2 and without surfactant.

Tests Nos. 7 and 8 are carried out according to the protocol given in example 1, with 1200 ppm of Erol 18 as surfactant.

Tests Nos. 9 to 11 are carried out according to the protocol given in example 2, with 1200 ppm of Erol 18 as surfactant.

The measurement of the index “b” is carried out on an average sample: a sample is taken every 12 hours, this being for 15 days, and then all of these samples taken are mixed together.

TABLE 1

| | | % recycling | | | Total |
|---|---|---|---|---|---|
| | | of the mother | Index | % | sugars |
| Tests | BV | liquors % | “b” | Yield | (ppm) |
| 1 | 50 | 0 | 1.80 | 81 | 58 |
| 2 | 100 | 0 | 2.50 | 80 | 98 |
| 3 | 50 | 50 | 4.20 | 89 | 158 |
| 4 | 50 | 0 | 1.75 | 80 | 41 |
| 5 | 100 | 0 | 2.10 | 79 | 59 |
| 6 | 50 | 50 | 3.90 | 90 | 157 |

TABLE 2

| | | % recycling | | | Total |
|---|---|---|---|---|---|
| | | of the mother | Index | % | sugars |
| Tests | BV | liquors | “b” | Yield | (ppm) |
| 7 | 300 | 0 | 0.71 | 82 | 4 |
| 8 | 300 | 50 | 0.91 | 90 | 16 |
| 9 | 300 | 0 | 0.65 | 81 | 2 |
| 10 | 300 | 50 | 0.87 | 90 | 10 |
| 11 | 300 | 60 | 0.95 | 94 | 16 |

The comparison between tables 1 and 2 clearly reveals the positive influence of the surfactant on the values of the index “b”.

In addition, it is possible to adjust both the value of this index and the yield of the process in terms of succinic acid, through the recycling of all or part of the first-crystallization mother liquors.

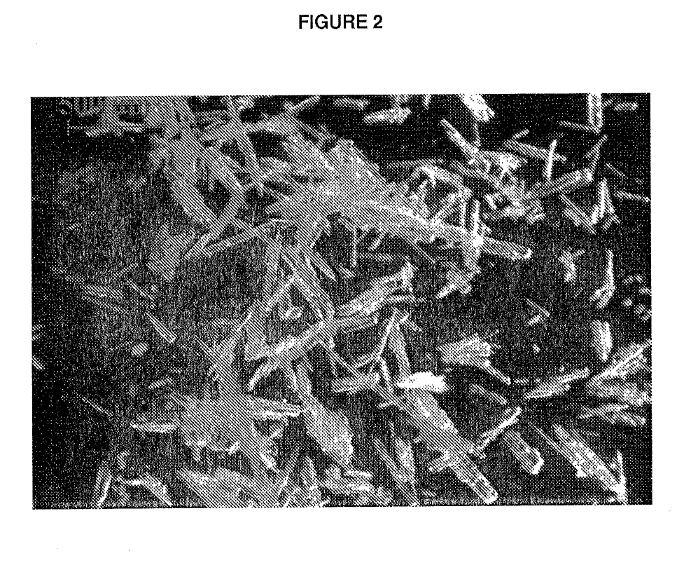

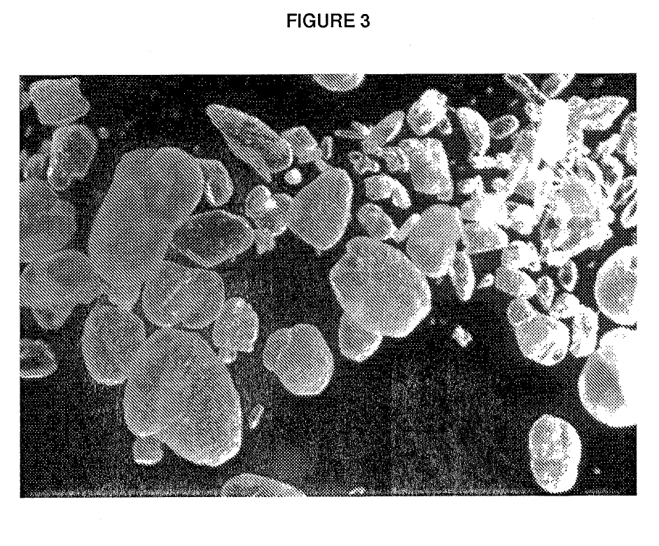

Finally, FIG. 2 reveals the morphology of the crystals obtained without surfactant (test No. 1), which are needles, whereas FIG. 3 (test No. 7) reveals balls, having an index much higher than 0.70. These photos were taken on a Leica EZ4HD microscope. This is particularly surprising insofar as it was known from document WO 01/07389 that the use of surfactants in a succinic acid crystallization process led to the formation of needle-shaped succinic acid crystals. As it happens, successfully achieving such ball-shaped crystals is particularly advantageous since, as previously indicated, the purification of the succinic acid is then improved for various reasons; moreover, these crystals flow better and have less of a tendency to cake. Without being bound by any theory, the applicant explains this fundamental difference in behavior during the crystallization by the impurities of a succinic acid obtained from a fermentation medium, which are different from those of the petroleum-based succinic acid of document WO 01/07389.

### Example 4

This example corresponds to the performing of tests outside the invention (with or without surfactant), in a single crystallization process.

During the various tests, crystals are sampled before the dissolving step c) in some of the tests of the previous example.

The crystals are then dried on a Retsch laboratory fluidized bed until a moisture content of 0.3% by weight of water, relative to the total weight of product, is obtained.

TABLE 3

| Tests | Index “b” |
|---|---|
| 1a | 9.01 |
| 7a | 5.05 |
| 4a | 8.27 |
| 9a | 4.39 |

The results of table 3 demonstrate that the processes with single crystallization, optionally in the presence of surfactant, do not result in satisfactory “b” values.

### Example 5

For tests Nos. 1, 4, 8 and 10, the stability of the process was tested by operating the process continuously over several days.

Samples were taken 24 hours apart for analysis. The “b” values thus determined are reproduced in table 4 below.

TABLE 4

| Days | Test 1 | Test 4 | Test 8 | Test 10 |
|---|---|---|---|---|
| 1 | 1.43 | 1.52 | 0.97 | 0.95 |
| 2 | 2.10 | 1.13 | 0.96 | 0.81 |
| 3 | 1.75 | 1.90 | 0.82 | 0.85 |
| 4 | 1.60 | 2.04 | 0.97 | 0.78 |
| 5 | 2.56 | 1.38 | 0.82 | 0.74 |
| 6 | 2.13 | 1.54 | 0.78 | 0.79 |
| 7 | 1.50 | 1.52 | 0.85 | 0.74 |
| 8 | 2.46 | 1.68 | 0.82 | 0.71 |
| 9 | 2.05 | 1.41 | 0.89 | 0.87 |
| 10 | 1.99 | 1.78 | 0.92 | 0.81 |
| 11 | 1.81 | 1.65 | 0.78 | 0.79 |
| 12 | 1.49 | 1.98 | 0.75 | 0.91 |
| 13 | 1.79 | 2.70 | 0.82 | 0.94 |
| 14 | 1.84 | 2.14 | 0.89 | 0.91 |
| 15 | 1.96 | 1.85 | 0.91 | 0.85 |
| Mean value | 1.92 | 1.75 | 0.86 | 0.83 |
| Standard deviation | 0.37 | 0.38 | 0.07 | 0.08 |

This table clearly establishes the stability of the process according to the invention, the measurements being much more reproducible than in the case of the prior art.

1. A process for producing succinic acid crystals from a fermentation medium containing succinic acid, comprising:
a) bringing a fermentation medium to a pH of between 1.0 and 4.0,
b) crystallizing succinic acid from the fermentation medium resulting from step to form first succinic acid crystals and a first crystallization mother liquor, then separating the first succinic acid crystals from the first crystallization mother liquors and then washing the obtained crystals with water to obtain washed succinic acid crystals,
c) dissolving the washed succinic acid crystals in water at a temperature between 30° C. and 70° C. to obtain a solution containing dissolved succinic acid,
d) purifying the solution by contacting said solution with activated carbon and on ion exchange resin,
e) crystallizing succinic acid contained in the purified solution to form second succinic acid crystals, and a second crystallization mother liquor, separating the second succinic acid crystals from the second crystallization mother liquors and then washing the second succinic acid crystals with water,
f) drying the washed, second succinic acid crystals of step e) to a moisture content of less than 0.5% and cooling them to a temperature below 30° C., wherein at least one surfactant is introduced before and/or during step b).

2. The process according to claim 1, wherein said surfactant comprises one of non-ionic surfactants comprising polysorbates having an HLB greater than 15 and alkylene oxide block copolymer based surfactants.

3. The process according to claim 1, comprising bringing the fermentation medium to a pH of between 1.5 and 3.5.

4. The process according to claim 1, wherein the fermentation medium includes one of bacteria comprising bacterial strains of the Mannheimia, Anaerobiospirillum, Bacillus or Escherichia genus, or from fungal strains.

5. The process according to claim 1, further comprising crystallizing succinic acid batchwise or continuously, by cooling.

6. The process according to claim 1, wherein the separating of steps b) and e) is conducted by filtration or centrifugation.

7. The process according to claim 1, wherein washing is carried out with water, at a temperature of between 15° C. and 25° C.

8. The process according to claim 1, wherein a part of at least one of said first and second crystallization mother liquors, and washing water is recycled as a feed to step b).

9. The process according to claim 1, wherein said dissolving provides a dry matter content of between 5% and 50% by weight of the total weight of said solution.

10. The process according to claim 1, wherein the activated carbon comprises powder or granules.

11. The process according to claim 1, wherein all of said second crystallization mother liquid sand washing mother liquors of step e) are recycled to step b).

12. The process according to claim 1, wherein said succinic acid crystals are dried to a moisture content of less than 0.4%, weight of water relative to the dry weight of succinic acid, and cooled to a temperature below 30° C.

13. Fermentation produced succinic acid crystals comprising a color index b, measured in the reference system L, a, b, of less than or equal to 1.00.

14. The succinic acid crystals according to claim 1 having a reducing sugar content of less than 20 ppm.

15. The succinic acid crystals according to claim 14, comprising a sphericity index, as measured according to the Rittenhouse test, at least equal to 0.70.

16. A composition containing succinic acid crystals prepared according to the method of claim 1.

17. Fermentatively produced succinic acid crystals having a moisture content of less than 0.5% by weight, a sphericity of greater than 0.70, and a color index b, measured in the reference system L, a, b, of less than or equal to 1.00.
